(19) United States
(12) Patent Application Publication
(10) Pub. No.: US 2021/0113387 A1
(43) Pub. Date: Apr. 22, 2021

(54) Toilet Training Composition

(71) Applicant: Dennis E Carmichael, Beaumont, CA (US)

(72) Inventor: Dennis E Carmichael, Beaumont, CA (US)

(21) Appl. No.: 16/659,045

(22) Filed: Oct. 21, 2019

(51) Int. Cl. A61F 13/42; C08L 91/06

(52) U.S. Cl. CPC A61F 13/42; C08L 91/06; A61F 2013/421; A61F 2013/422; A61F 2013/427

(57) ABSTRACT

A toilet training composition is disclosed comprising a substrate provided as a folded arrangement and dispensable in a fluid reservoir. An additive is provided to the substrate which includes an indicator responsive to a change in at least one condition.

### TECHNICAL FIELD

The embodiments generally relate to colorant systems that can be triggered by the presence of an aqueous substance and more specifically relates to a colorant that can be activated or deactivated by pH conditions so as to provide a visually observable signal.

### BACKGROUND

Many products can be used more effectively if they include a feature that provides a feedback for a particular input. Many manufacturers incorporate color-changing dyes into a product such that the product changes colors in response to a stimulus. For example, color-changing dyes have been added to clothing to respond to contact with a liquid. Similarly, color-changing dyes may respond to changes in temperature using a special ink known as thermochromic ink.

These color-changing responses often provide an increase in satisfaction and enhance the consumer's experience of the products by relaying real-time feedback to inform the user of changing conditions.

Color-changing technologies have been described in the arts. Color indicators can either indicate a change in condition or a degree of use through a change from “no color” to “color” or through a change from one color to another.

Similarly, effervescent compositions are well known in the arts of chemistry. Such chemicals release a gas when in the presence of a fluid or other chemical. Effervescent chemicals also provide a feedback mechanism which may incentivize the user to behave in a certain manner.

Toilet training a child can be difficult and frustrating for parents and children alike. Many training aids, including training devices and instructions for training, exist in the market, however, many fall short in expediting the toilet training process.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments provided herein relate to a toilet training composition comprising a substrate provided as a folded arrangement and dispensable in a fluid reservoir. An additive is provided to the substrate which includes an indicator responsive to a change in at least one condition.

The toilet training composition provides a reward for a child during toilet training by providing feedback once the composition is in the presence of urine. The reward may help the learning process which is inherently difficult for children and parent alike by providing visual feedback for the proper toilet trained behavior.

In one aspect, the additive is comprised of an effervescent substance.

In one aspect, the additive is comprised of a color indicator.

In one aspect, the additive is activated by a change in the pH of a fluid.

In one aspect, the additive is inactive between a pH of about 6.5-8.5.

In one aspect, the additive is active at a pH below 6.5.

In one aspect, the additive is activated by the presence of ammonia in the fluid.

In one aspect, the substrate is a packed tissue arranged to unfold into a shape in the presence of urine.

In one aspect, the substrate is a packed granular composition.

In one aspect, the packed granular composition is a soluble carbohydrate.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a perspective view of the toilet training device, according to some embodiments; and

FIG. 2 illustrates a perspective view of the toilet training device, according to some embodiments.

### DETAILED DESCRIPTION

The specific details of the single embodiment or variety of embodiments described herein are to the described device and methods of use. Any specific details of the embodiments are used for demonstration purposes only, and no unnecessary limitations or inferences are to be understood therefrom.

Before describing in detail exemplary embodiments, it is noted that the embodiments reside primarily in combinations of components of the device. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to a toilet training composition which may be added to the fluid contained in a toilet. The toilet training composition is disposed within the toilet before the user urinates in the toilet bowl. The toilet training composition is provided with an additive which includes an indicator that is responsive to a change in condition of the fluid in the toilet.

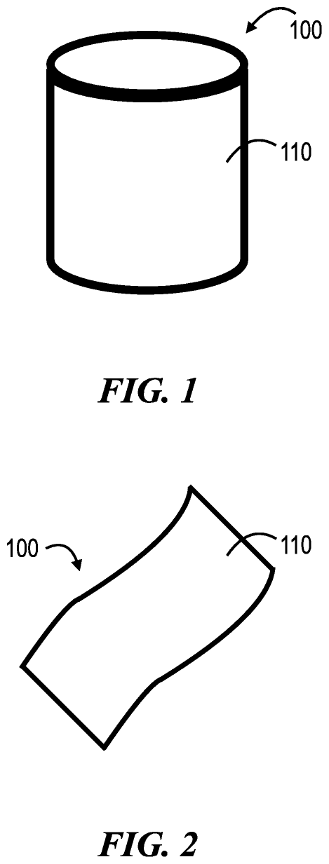

FIG. 1 illustrates the toilet training composition 100 having a substrate 110 arranged as a shape. The substrate 110 is provided with an indicator which may include a color indicator or an effervescent chemical which is activated in the presence of urine.

FIG. 2 illustrates the toilet training composition 100 having a substrate 110 arranged as a shape. The substrate 110 is formulated to dissolve in the presence of urine to release the additive provided with the substrate.

In some embodiments, the additive comprises a color indicator which reacts to the presence of urine to cause a change in the color of the fluid contained in the toilet bowl. For example, the color indicator may be thermally activated upon a change in temperate to the fluid once urine is deposited into the toilet bowl. The addition of urine may raise the temperature of the fluid to a temperature which causes a change in color, providing feedback to the child being toilet trained.

In some embodiments, the additive reacts to the presence of ammonia. One skilled in the arts will readily understand chemicals which react to ammonia in order to produce a change in color, an effervescent reaction, or other reaction which provides feedback during toilet training.

The additive may include any known chemicals which react to the presence of urine, such as chemicals which react to the presence of chemicals present in urine such as ammonia, to the change in pH due to the deposit of urine into the fluid of the toilet bowl, or the change in temperature caused by the deposit of urine into the fluid of the toilet bowl.

The substrate may be provided to the user in various shapes. Shapes which may appeal to a child may be beneficial during the toilet training process by providing an incentive to urinate in the toilet. The substrate may provide an incentive for “aiming” correctly into the toilet bowl during toilet training by providing feedback when the urine is accurately disposed into the toilet bowl.

In some embodiments, the substrate is comprised of paraffin wax which unfolds in the presence of a fluid, such as when in the presence of urine, to result in a reward during toilet training.

Effervescent additives which are provided with the substrate may include sodium bicarbonate, carbon dioxide, nitrogen gas, or other known chemical compositions which have effervescent reactions in the presence of a fluid.

Color indicators may be activated by a change in temperature, a change in pH, or by the presence of a chemical (such as ammonia). One skilled in the arts will readily understand that various color indicators may be provided with the substrate to provide feedback during toilet training.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A toilet training composition, comprising:
a substrate provided as a folded arrangement and dispensable in a fluid reservoir;
an additive provided to the substrate, the additive including an indicator responsive to a change in at least one condition.

2. The composition of claim 1, wherein the additive is comprised of an effervescent substance.

3. The composition of claim 1, wherein the additive is comprised of a color indicator.

4. The composition of claim 1, wherein the additive indicator is activated by a change in pH of a fluid.

5. The composition of claim 4, wherein the additive is inactive between a pH of about 6.5-8.5.

6. The composition of claim 5, wherein the additive is active at a pH below 6.5.

7. The composition of claim 5, wherein the additive is activated by the presence of ammonia in a fluid.

8. The composition of claim 1, wherein the substrate is a packed tissue arranged to unfold into a shape in the presence of urine.

9. The composition of claim 1, wherein the substrate is a packed granular composition.

10. The composition of claim 9, wherein the packed granular composition is a soluble carbohydrate.

11. A toilet training composition, comprising:
a biodegradable substrate provided as a folded arrangement and dispensable in a fluid reservoir;
an additive provided to the substrate, the additive including an indicator responsive the presence of ammonia when disposed in a toilet.

12. The composition of claim 11, wherein the biodegradable substrate is paraffin wax.

13. The composition of claim 11, wherein the additive is comprised of an effervescent substance.

14. The composition of claim 11, wherein the additive is comprised of a color indicator.

15. The composition of claim 11, wherein the additive indicator is activated by a change in pH of a fluid disposed in the toilet.

16. The composition of claim 15, wherein the additive is inactive between a pH of about 6.5-8.5.

17. The composition of claim 16, wherein the additive is active at a pH below 6.5.

18. The composition of claim 17, wherein the additive is activated by the presence of ammonia in a fluid.

19. The composition of claim 18, wherein the at least one condition is a change in temperature.

20. A toilet training composition, comprising:
a granular carbohydrate substrate provided in a formed shape and dispensable in a fluid reservoir;
an additive provided to the substrate, the additive including an indicator responsive to a change in at least one condition when disposed in a toilet.
